(19) United States
(12) Patent Application Publication
(10) Pub. No.: US 2019/0247309 A1
(43) Pub. Date: Aug. 15, 2019

(54) SELF-EMULSIFYING LIPID COMPOSITIONS

(71) Applicant: FRIULCHEM, Vivaro (IT)

(72) Inventors: Guy Derrieu, Cagnes Sur Mer (FR); Disma Giovanni Mazzola, Milan (IT); Giancarlo Mazzola, Bedano (CH)

(73) Assignee: FRIULCHEM, Vivaro (IT)

(21) Appl. No.: 16/308,615

(22) PCT Filed: Jun. 7, 2017
(86) PCT No.: PCT/EP2017/063871

§ 371 Date: Dec. 27, 2018

(30) Foreign Application Priority Data
Jun. 10, 2016 (FR) 1600945

(51) Int. Cl. A61K 9/107; A61K 31/165; A61K 47/20; A61K 47/26; A61K 47/14; A61K 47/38; A61K 47/22; A61K 9/00; A61K 31/365; A61K 47/36; A61K 31/277

(52) U.S. Cl. CPC A61K 9/107; A61K 31/165; A61K 47/20; A61K 47/26; A61K 47/14; A61K 47/38; A61K 9/1075; A61K 9/0095; A61K 9/0053; A61K 31/365; A61K 9/0017; A61K 47/36; A61K 31/277; A61K 47/22

(57) ABSTRACT

The invention concerns a lipid composition that is self-emulsifying in the presence of a hydrophilic phase, comprising between 0.001% and 20% by weight, relative to the total weight of the composition, of at least one active substance; between 5% and 60% by weight, relative to the total weight of the composition, of an oily phase; a first surfactant; a second surfactant; the ratio between the first surfactant and the second surfactant being between 0.5 and 6; said lipid composition is such that, after dispersion in the hydrophilic phase, it forms particles having, as an absolute value, a charge of between 10 mV and 100 mV. The invention also concerns a method for preparing a self-emulsifying lipid composition, and the use of said self-emulsifying lipid composition in the pharmacutical or nutraceutical veterinary field.

### FIELD OF THE INVENTION

The present invention relates to the field of preparing self-emulsifying lipid compositions in particular for therapeutic or nutraceutical use. These compositions are intended for the treatment of animals and/or plants. The invention more specifically relates to a self-emulsifying lipid composition in the presence of a hydrophilic phase and which has, after dispersion in said hydrophilic phase, electrically charged particles. The invention also relates to a process for preparing said composition, as well as uses of an emulsion, microemulsion or nanoemulsion obtained from said composition.

### PRIOR ART

The oral and topical routes are the preferred routes for administration of drug or nutraceutical active substances to animals. Nevertheless, these active substances very often have low water-solubility or, more generally, in hydrophilic phases, are even insoluble in such phases. In some cases, they even have a certain instability in the presence of hydrophilic phases. These are the reasons for which scientists developed different processes and means for facilitating and optimizing the administration of an active substance to animals.

Document FR 2 761 912 describes a process for adhesion of an active substance to a living surface or an inert surface, that implements multilamellar microcapsules in a water-surfactant medium or polar-surfactant solvent. These multilamellar vesicles are positively charged owing to the use of a cationic surfactant or a cationic polymer. They therefore have a certain adherence to living or inert surfaces.

Document FR 2 761 886 describes a composition containing at least one cationic polymer, and at least one active substance contained in a micro or nanoparticle vector. According to said document, the choices of the micro or nanoparticle vector, the cationic polymer and the particular association of the two make it possible to modulate the activity of the composition. This composition is then dispersed in an aqueous phase allowing for a treatment extended over time. It should be noted that these micro or nanoparticle vectors are not emulsions. The production of these vectors is complex. The compositions disclosed in said prior art do not belong to the field of self-emulsifying lipid compositions.

Document FR 2 790 404 describes compositions for the treatment or maintenance of aquatic environments, or for the treatment or maintenance of one or more targets contained in aquatic environments. These compositions include at least one active molecule contained in at least one micro or nanoparticle vector. These vectors also contain a cationic polymer. These compositions are used by being dipped in the environment to be treated. Depending on the intended target, the composition has higher or lower density. For example, a composition having a high density with respect to density of water will make it possible to treat the floor of aquatic environments. A composition having a density on the same order of magnitude as that of water will make it possible to treat living beings, for example, fish moving in aquatic environments, by dipping.

In addition, numerous studies have been conducted for the implementation of emulsions, microemulsions or nanoemulsions obtained from self-emulsifying lipid formulations incorporating active substances. These self-emulsifying lipid formulations are also called SELF for self-emulsifying lipid formulations.

These SELF formulations can integrate a range of functional lipid excipients such as oily vehicles, surfactants or co-solvents. Due to their specific compositions, these excipients are capable of forming an emulsion in contact with an aqueous solution when they are used alone or with an active substances. Typically, these excipients are chosen by means of a specific excipient-active substance selection making it possible to identify the compatible excipients capable of offering the greatest solubility of the active substance.

SELF formulations have been studied widely and used in various applications, in particular in human and veterinary pharmacy or in cosmetic applications.

For example, SELF formulations have been used for the treatment of animals via the oral route, using drinking water as a vehicle. In practice, the introduction of an active molecule into the drinking water of animals requires the use of a concentrated aqueous solution generally distributed by means of a dosing pump in the water circulation circuit. Document FR 2 925 855 proposes a self-emulsifying liquid formulation including an active molecule, florfenicol, capable of being subjected to an aqueous dilution, having an active molecule content greater than its water-solubility limit. The solution provided by said document is not entirely satisfactory because the dilution in the aqueous phase leads to an emulsion that is not stable for at least 24 hours, in every case of use. This instability leads to a certain heterogeneity in the dilution of the self-emulsifying liquid formulation in the hydrophilic phase. A phase shift is observed after 6 hours, leading to poor distribution of the active molecule to the animals and causing clogging of various elements of the distribution circuit.

In another example, SELF formulations have been used for the treatment of animals or plants, via a topical application, in this case, the emulsions are applied to living surfaces such as the skin or coats of animals, or on the foliage or cuticle of plants. These emulsions can also be applied to so-called inert surfaces such as natural or artificial fibers, or to fiber-based products such as fabric. In particular, documents U.S. Pat. No. 5,968,990 and U.S. Pat. No. 6,255,350 propose formulations capable of forming microemulsions by dispersion in water. These microemulsions are prepared by using non-ionic surfactants. The dispersions obtained are vaporized, poured or used by dipping in order to deposit active ingredients on living surfaces, or even, in some embodiments disclosed in said documents, on inert surfaces. However, these active ingredients carried in hydrophobic droplets dispersed in water are deposited without adhering to the aforementioned surfaces. The aqueous vehicle thus carries some of said active ingredients, which are ultimately lost and pollute the environment. If the deposition on the target surface is performed in rainy conditions, or if it rains immediately after such a deposition, the active ingredients are washed away.

### SUMMARY OF THE INVENTION

In consideration of the above-mentioned prior art, one problem to be solved by the invention is that of producing a self-emulsifying lipid composition in the presence of a hydrophilic phase that includes an active substance, and that makes it possible to overcome the aforementioned known problems of the prior art.

The solution proposed according to the invention to this stated problem has, as its first object, a self-emulsifying lipid composition in the presence of a hydrophilic phase, including:

between 0.001% and 20% by weight, relative to the total weight of the composition, of at least one active substance;

between 5% and 60% by weight, relative to the total weight of the composition, of an oily phase;

a first surfactant;

a second surfactant;

between 0.5% and 20% by weight with respect to the total weight of the composition of at least one polymer soluble in the composition and/or an ionic polymer insoluble in the composition;

in which the ratio between the first surfactant and the second surfactant is between 0.5 and 6;

according to which said lipid composition is such that, after dispersion in the hydrophilic phase, it forms particles having, as an absolute value, a charge of between 10 mV and 100 mV.

In particular, these self-emulsifying lipid compositions make it possible, once dispersed in the hydrophilic phase, to optimize the administration of veterinary pharmaceutical or nutraceutical treatments or treatments for plants. They also make it possible, once dispersed in the hydrophilic phase, to limit the phenomenon of clogging in drinking water distribution networks, as well as the phenomenon of pollution due to losses of the active substances in nature.

The invention has, as a second object, a method for preparing a self-emulsifying lipid composition, including the following steps:

an active substance is provided;

an oily phase is provided;

a first surfactant is provided;

a polymer soluble in the composition and/or an ionic polymer insoluble in the composition is provided;

a second surfactant is provided; then the active substance, the oily phase, the first and the second surfactant, and a soluble polymer and/or an insoluble ionic polymer are mixed under agitation, with or without heating.

It has, as a third object, a self-emulsifying lipid composition as defined above, forming electrically charged particles after dispersion of said composition in the hydrophilic phase, for use as a drug in the pharmaceutical and/or dermatological treatment of animals.

It has, as a fourth object, a self-emulsifying lipid composition as defined above, forming, after dispersion in the hydrophilic phase, positively charged particles for use as a drug for topical application on the skin, coat or appendages of animals in the treatment of a disease.

It has, as a fifth object, a self-emulsifying lipid composition as defined above, forming, after dispersion in the hydrophilic phase, negatively charged particles for use as a drug for oral administration of a pharmaceutical treatment in animals.

It has, as a sixth object, a self-emulsifying lipid composition as defined above, forming electrically charged particles after dispersion of said composition in the hydrophilic phase for the nutraceutical treatment of animals.

It has, as a seventh object, a self-emulsifying lipid composition as defined above, forming electrically charged particles after dispersion of said composition in the hydrophilic phase for the treatment of plants.

It has, as an eight object, a self-emulsifying lipid composition as defined above, forming, after dispersion in the hydrophilic phase, negatively charged particles, for oral administration in the nutraceutical treatment of animals.

Advantageously, the active substance is chosen from anti-infectious agents such as antibiotics and sulfamides; cardiotonics; antiparasitic agents; insecticides; insect growth inhibitors; anti-arthritic agents; steroidal or nonsteroidal anti-inflammatories; antihistamines; hormones such as prostaglandins; digestive therapy substances such as gastrointestinal dressings and sedatives, antiulcer agents and replacement flora, anti-diarrheal agents, hepatoprotective agents, antispasmodic agents, laxatives, intestinal antiseptics; respiratory therapy substances such as respiratory analeptics, cough suppressants, bronchodilators, mucolytics, and respiratory antiseptics; substances acting on the nervous system such as analgesics, sedatives and tranquilizers; macroelements, microelements and trace elements; vitamins; plant or animal organ extracts; and mixtures thereof; —the first surfactant and the second surfactant are chosen from: anionic surfactants such as alkyl sulfates, alkyl ether sulfates, amidoalkyl ether sulfates, aryl alkyl polyether sulfates, monoglyceride sulfates, alkyl sulfonates, amidoalkyl sulfonates, aryl alkyl sulfonates, alpha olefin sulfonates, paraffin sulfonates, alkyl sulfosuccinates, ether alkyl sulfosuccinates, amidoalkyl sulfosuccinates, alkyl sulfoacetates, acyl sarcosinates, acyl glutamates, alkyl sulfosuccinamates, acyl thionates, N-acyl laurate, alkyl monoester salts and polycarboxylic polyglycoside acids, acyl lactylates, D-galactoside uronic acid salts, carboxylic alkyl ether acid salts, carboxylic alkyl aryl ether acid salts, carboxylic alkyl ether amido acid salts; cationic surfactants such as amine ethoxylates such as bis (2-hydroxylethyl) coco alkylamines, bis (2-hydroxylethyl) soya alkylamines, bis (2-hydroxyethyl) tallowalkylamines, polyoxyethylene (15) soya alkylamines; amine salts such as ricinoleamidopropyl dimethylamine lactate; protein derivatives such as N-[2-hydroxy-3-(lauryldimethylamino) propyl] hydrolyzed collagen chloride; quaternary ammonium salts such as methyl sulfate of imidazolium compounds of dihydro-4,5 methyl-1 tallow alkyl nor-2 (tallow amidoethyl-2)-1, 2-[bis (2-hydroxypropyl) amino]ethyl] bis (2-hydroxypropyl) (methyl) ammonium methyl sulfate, dioleate (ester), lauryl methyl gluceth-10 hydroxypropyl dimonium chloride, behentrimonium chloride, polyglycolether (15 EO) coco ammonium methosulphate; non-ionic surfactants such as hydrogenated castor oil polyethers and ethylene oxide such as hydrogenated polyethylene-40 castor oil, hydrogenated polyethylene-60 castor oil; fatty acid and sorbitan esters such as sorbitan monolaurate, sorbitan monostearate; polyoxyethylated fatty acid and sorbitan esters such as polyoxyethylene (20) sorbitan monolaurate, polyoxyethylene (20) sorbitan monopaImitate, polyoxyethylene (20) sorbitan monostearate; fatty acid and polyethylene glycol esters such as PEG-20 stearate, PEG-32 stearate; fatty acid and polyoxylglyceride esters such as caprylocaproyl polyoxyl-8 glycerides, lauroyl macrogol-32 glycerides, stearoyl macrogol-32 glycerides; polyglycerol fatty acid esters such as polyglyceryl-3 oleate, polyglyceryl-6 dioleate, polyglyceryl-6 isostearate; —the oily phase is chosen from plant-based oils such as palm oil, sunflower oil, rapeseed oil, castor oil, peanut oil, corn oil; saturated or unsaturated fatty acid esters including fatty acids with 8 to 18 carbon atoms and including hydrocarbon chains with 1 to 12 carbon atoms such as isopropyl myristate, isopropyl palmitate, butyl stearate, hexyl laurate, isocetyl palmitate, isocetyl stearate, isostearyl isostearate; fatty alcohol ethers such as distearyl ether; triglycerides, diglycerides, monoglycerides and mixtures thereof such as glycerol monooleate, glycerol monolinoleate; mixtures of mono-, di-, and triglycerides including a fatty acid with 8 to 18 carbon atoms and polyethylene glycol diesters; —the polymer is chosen from: anionic polymers such as anionic derivatives of starch such as carboxymethyl sodium starch, sodium starch octenylsuccinate, anionic derivatives of cellulose such as sodium carboxymethylcellulose, xanthan gum, carbomers, methacrylic acid and methyl methacrylate copolymers; cationic polymers such as copolymers of dimethylaminoethyl methacrylate, butyl methacrylate, and methyl methacrylate, copolymers of vinylpyrrolidone and substituted quaternized vinylpyrrolidone, butyl acrylate chloride/ethyltriminium/styrene, poly(diallyldimethylammonium) chloride, acrylamide and diallyldimethylammonium chloride copolymers, hydroxypropyl guar hydroxypropyltrimonium chloride, guar hydroxypropyltrimonium chloride, modified xanthan gums, chitosan and derivatives thereof; non-ionic polymers such as cellulose ethers such as methylcellulose, ethylcellulose, hydroxyethyl ethyl cellulose, hydroxypropyl cellulose, hydroxypropyl methyl cellulose; —the polymer soluble in the composition and/or the ionic polymer insoluble in the composition represents 1% to 15% by weight of the total weight of the composition; —the self-emulsifying lipid composition also includes at least one additive chosen from the hydrophilic solvents, preservatives, sweetening agents and appetizing agents; and—additives representing 0.01% to 30% by weight with respect to the total weight of the composition; —the method for preparing the self-emulsifying lipid composition also includes, prior to mixing the various compounds, the following steps: a ternary phase diagram is drawn, including the compounds of the self-emulsifying lipid composition and the hydrophilic phase; then, on the basis of said ternary diagram, the proportions of the various compounds of the self-emulsifying lipid composition are selected; then the self-emulsifying lipid composition is prepared by mixing, with or without heating, under agitation, the various compounds of the self-emulsifying lipid composition.

### DETAILED DESCRIPTION OF THE INVENTION

The invention will be better understood after reading the following non-limiting description.

The invention concerns self-emulsifying lipid compositions that, after dispersion in a hydrophilic phase, form electrically charged particles.

These compositions include at least one active substances, one oily phase, one first surfactant and one second surfactant.

The term “active substance” refers to a pharmaceutical or nutraceutical substance having a therapeutic effect or having a biological activity.

The active substance(s) is (are) present, in the composition according to the invention, in an amount of between 0.001% and 20% by weight with respect to the total weight of said composition, advantageously between 2% and 16% by weight with respect to the total weight of said composition, and even more advantageously between 4% and 12% by weight with respect to the total weight of said composition.

Advantageously, the active substance is chosen from anti-infectious agents such as antibiotics and sulfamides; cardiotonics; antiparasitic agents; insecticides; insect growth inhibitors; anti-arthritic agents; steroidal or nonsteroidal anti-inflammatories; antihistamines; hormones such as prostaglandins; digestive therapy substances such as gastrointestinal dressings and sedatives, antiulcer agents and replacement flora, anti-diarrheal agents, hepatoprotective agents, antispasmodic agents, laxatives, intestinal antiseptics; respiratory therapy substances such as respiratory analeptics, cough suppressants, bronchodilators, mucolytics, and respiratory antiseptics; substances acting on the nervous system such as analgesics, sedatives and tranquilizers; macroelements, microelements and trace elements; vitamins; plant or animal organ extracts; and mixtures thereof.

In a preferred embodiment, the active substance is chosen from antibiotics such as florfenicol, tiamulin, valnemulin, and bicozamycin. In another embodiment, the active substance is chosen from antiparasitic agents such as ivermectin, moxidectin, milbemycin, emamectin and derivatives thereof such as benzoate, pyrantel, and derivatives thereof such as pamoate, praziquantel, benzimidazoles, salts thereof and derivatives thereof. In an advantageous embodiment, the active substance is chosen from insecticides such as fampronil, fipronil, cypermethrin, deltamethrin, teflubenzuron, diflubenzuron, azamethiphos, and pyriproxyfen.

Preferably, the nutraceutical active substance is chosen from plant or animal organ extracts. These plant or animal organ extracts are advantageously chosen for their anti-infectious, antibacterial, antifungal, anti-diarrheal, hepatoprotective, antispasmodic/laxative, or intestinal antiseptic action. In another embodiment, these plant or animal organ extracts are chosen for their actions on respiratory problems such as cough, as bronchodilators, mucolytics, and respiratory antiseptics. In another embodiment, the nutraceutical active substance is chosen from analgesics, sedatives, tranquilizers, anti-arthritic agents, insecticides, antiparasitic agents, anti-ulcer agents, and anti-stress agents. Advantageously, replacement flora; macroelements, microelements and trace elements; vitamins; and mixtures thereof are also used as nutraceutical active substances.

The self-emulsifying lipid compositions according to the invention also include an oily phase.

The oily phase is present in the composition according to the invention in an amount of between 5% and 60% by weight with respect to the total weight of said composition, preferably between 1% and 30% by weight with respect to the total weight of said composition, and even more preferably between 10% and 25% by weight with respect to the total weight of said composition.

In the compositions according to the invention, the oily phase is an oily compound commonly used for pharmaceutical, dermatological or nutraceutical applications.

The oily phase is advantageously chosen from plant-based oils such as palm oil, sunflower oil, rapeseed oil, castor oil, peanut oil, and corn oil; saturated or unsaturated fatty acid esters including fatty acids with 8 to 18 carbon atoms and including hydrocarbon chains with 1 to 12 carbon atoms such as isopropyl myristate, isopropyl palmitate, butyl stearate, hexyl laurate, isocetyl palmitate, isocetyl stearate, isostearyl isostearate; fatty alcohol ethers such as distearyl ether; triglycerides, diglycerides, monoglycerides and mixtures thereof such as glycerol monooleate, glycerol monolinoleate; mixtures of mono-, di-, and triglycerides including a fatty acid with 8 to 18 carbon atoms and polyethylene glycol diesters; saturated or unsaturated fatty acid esters including fatty acids with 6 to 18 carbon atoms and propylene glycol such as propylene glycol monocaprylate, propylene glycol monolaurate, propylene glycol dilaurate, and propylene glycol dioleate.

The lipid and self-emulsifying compositions according to the invention include a first surfactant and a second surfactant.

The first surfactant and the second surfactant are advantageously chosen from:

anionic surfactants such as alkyl sulfates, alkyl ether sulfates, amidoalkyl ether sulfates, aryl alkyl polyether sulfates, monoglyceride sulfates, alkyl sulfonates, amidoalkyl sulfonates, aryl alkyl sulfonates, alpha olefin sulfonates, paraffin sulfonates, alkyl sulfosuccinates, ether alkyl sulfosuccinates, amidoalkyl sulfosuccinates, alkyl sulfoacetates, acyl sarcosinates, acyl glutamates, alkyl sulfosuccinamates, acyl thionates, N-acyl laurate, alkyl monoester salts and polycarboxylic polyglycoside acids, acyl lactylates, D-galactoside uronic acid salts, carboxylic alkyl ether acid salts, carboxylic alkyl aryl ether acid salts, carboxylic alkyl ether amido acid salts;

cationic surfactants such as amine ethoxylates such as bis (2-hydroxylethyl) coco alkylamines, bis (2-hydroxylethyl) soya alkylamines, bis (2-hydroxyethyl) tallowalkylamines, polyoxyethylene (15) soya alkylamines; amine salts such as ricinoleamidopropyl dimethylamine lactate; protein derivatives such as N-[2-hydroxy-3-(lauryldimethylamino) propyl] hydrolyzed collagen chloride; quaternary ammonium salts such as methyl sulfate of imidazolium compounds of dihydro-4,5 methyl-1 tallow alkyl nor-2 (tallow aminoethyl-2)-1, 2-[bis (2-hydroxypropyl) amino] ethyl] bis (2-hydroxypropyl) (methyl) ammonium methyl sulfate, dioleate (ester), lauryl methyl gluceth-10 hydroxypropyl dimonium chloride, behentrimonium chloride, polyglycolether (15 EO) coco ammonium methosulphate;

non-ionic surfactants such as hydrogenated castor oil polyethers and ethylene oxide such as hydrogenated polyethylene-40 castor oil, hydrogenated polyethylene-60 castor oil; fatty acid and sorbitan esters such as sorbitan monolaurate, sorbitan monostearate; polyoxyethylated fatty acid and sorbitan esters such as poiyoxyethylene (20) sorbitan monolaurate, polyoxyethylene (20) sorbitan monopalmitate, polyoxyethylene (20) sorbitan monostearate; fatty acid and polyethylene glycol esters such as PEG-20 stearate, PEG-32 stearate; fatty acid and polyoxylglyceride esters such as caprylocaproyl polyoxyl-8 glycerides, lauroyl macrogol-32 glycerides, stearoyl macrogol-32 glycerides; polyglycerol fatty acid esters such as polyglyceryl-3 oleate, polyglyceryl-6 dioleate, and polyglyceryl-6 isostearate.

In one embodiment, the first surfactant is water-soluble.

In another embodiment, the second surfactant is water-insoluble.

The ratio between the first surfactant and the second surfactant is between 0.5 and 6, and this ratio is preferably between 1 and 4.

In a preferred embodiment, the self-emulsifying lipid compositions according to the invention also include at least one polymer.

The polymer is advantageously present in an amount of between 0.5% and 20% by weight with respect to the total weight of the composition, preferably between 1% and 15% by weight with respect to the total weight of the composition, and even more preferably between 2.5% and 15% by weight with respect to the total weight of the composition.

The polymer according to the invention is preferably chosen from:

anionic polymers such as anionic derivatives of starch such as carboxymethyl sodium starch, sodium starch octenylsuccinate, anionic derivatives of cellulose such as sodium carboxymethylcellulose, xanthan gum, carbomers, methacrylic acid and methyl methacrylate copolymers;

cationic polymers such as copolymers of dimethylaminoethyl methacrylate, butyl methacrylate, and methyl methacrylate, copolymers of vinylpyrrolidone and substituted quaternized vinylpyrrolidone, butyl acrylate chloride/ethyltrimonium/styrene, poly(diallyidimethylammonium) chloride, acrylamide and diallyldimethylammonium chloride copolymers, hydroxypropyl guar hydroxypropyitrimonium chloride, guar hydroxypropyltrimonium chloride, modified xanthan gums, chitosan and derivatives thereof;

non-ionic polymers such as cellulose ethers such as methylcellulose, ethylcellulose, hydroxyethyl ethyl cellulose, hydroxypropyl cellulose, hydroxypropyl methyl cellulose.

Advantageously, the polymer is soluble in said composition. The term “soluble polymer” means that it must include a polymer soluble in the composition and which therefore is located in the particle during dispersion of the composition according to the invention in the hydrophilic phase.

According to another embodiment, the polymer is insoluble in said composition. The term “insoluble polymer” means that it must include a polymer insoluble in the composition and which therefore, on the one hand, is in suspension in the composition, and, on the other hand, forms a hydrated network in contact with the hydrophilic phase, i.e. a gel around electrically charged particles during the dispersion of the composition according to the invention in the hydrophilic phase. Advantageously, the insoluble polymer is ionic.

In another embodiment, the composition includes a combination of two polymers, one soluble in the self-emulsifying lipid composition and the other insoluble in said composition.

The self-emulsifying lipid composition according to the invention forms, after dispersion in the hydrophilic phase, electrically charged particles having, as an absolute value, a charge of between 10 mV and 100 mV, advantageously between 10 mV and 85 mV, and even more advantageously between 10 mV and 75 mV.

The composition according to the invention leads to a dispersion of electrically charged particles in a hydrophilic phase. Thus, a person skilled in the art will make sure that at least one compound of the composition carries and electric charge and that it is sufficient for conferring the desired charge on the particles.

The electric charge is advantageously carried either by the first surfactant or by the second surfactant.

In a preferred embodiment, the electric charge is carried by the polymer(s). In another preferred embodiment, the electric charge is carried either by the soluble polymer or by the insoluble polymer.

The self-emulsifying lipid composition according to the invention may also include at least one additive. Advantageously, the additive is) according to the invention is (are) present in an amount of between 0.01% and 30% by weight with respect to the total weight of the composition, and preferably between 0.3% and 20% by weight with respect to the total weight of the composition.

The additive(s) is (are) advantageously chosen from hydrophilic solvents, preservatives, pH regulators, sweetening agents, and appetizing agents.

The term “hydrophilic solvents” means that it must entirely water-soluble solvents. The hydrophilic solvents according to the invention are preferably chosen from alcohols, ethanol, isopropanol, polyols, glycerol, propylene glycol, polyethylene glycols such as PEG 200, PEG 300, PEG 400, PEG 600, glycol ethers such as diethylene glycol monomethyl ether, propylene glycol monomethyl ether, dipropylene glycol monomethyl ether, pyrrolidine-2-one, N-methyl pyrrolidone, N-substituted derivatives of pyrrolidine-2-one, and dimethylacetamide.

The hydrophilic solvent may advantageously be added to the composition according to the invention, in order to solubilize the active substance.

The preservatives according to the invention are advantageously chosen from derivatives of ascorbic acid, butylhydroxyanisol, butylhydroxytoluene, gallic acid and derivatives thereof such as propyl gallate and mixtures thereof.

The pH regulators according to the invention are advantageously chosen from citric acid and δ-glucono lactone.

The choice of proportions of the components of the formulation is made using a pseudo-ternary phase diagram, established according to techniques well known to a person skilled in the art, oily phase/first surfactant ratio: second surfactant/water. This diagram has at least one area for existence of an emulsion, microemulsion or nanoemulsion oil-in-water phase. The concentrations of the constituents of the composition are chosen so that it is possible to reach said emulsion, microemulsion or nanoemulsion area by simple dilution in the hydrophilic phase.

In other words, said diagram allows for an optimal choice of the proportions of each constituent of the self-emulsifying lipid composition. This optimal choice of the proportions of the constituents of the composition according to the invention thus enables a person skilled in the art to produce an emulsion, a microemulsion or a nanoemulsion.

The term “emulsion” refers to a mixture of two immiscible liquids. It more specifically concerns a dispersion of droplets of a lipophilic or hydrophobic liquid in another hydrophilic liquid, such as, for example, an oil-in-water emulsion, which leads to a system that is stable over a longer or shorter time associated with the self-emulsifying lipid composition. An emulsion generally has a white appearance. When the droplet size is smaller than 200 nm, the system is described as a microemulsion. When the droplet size is smaller than 100 nm, the system is described as a nanoemulsion. The microemulsion and the nanoemulsion are transparent and thermodynamically stable systems.

The self-emulsifying lipid composition according to the invention is present either in the form of a solution or in the form of a suspension, if an insoluble polymer is used in said composition. Whether it is a solution or a suspension, it is desirable to obtain a fluid self-emulsifying lipid composition so that the auto-emulsion forms by means of simple manual agitation. The fluidity of the composition and the agitation system used influence the dispersion of said composition in the hydrophilic phase as well as the size of the particles obtained.

Depending on the intended use, a person skilled in the art may choose the particle size according to the shear force applied, i.e. according to the agitation used, manual or mechanical, to form the emulsion, the microemulsion or the nanoemulsion.

According to one embodiment, the self-emulsifying lipid composition is in the form of a gel. In this case, a significant shear force is applied to disperse the hydrophilic phase and obtain an emulsion.

According to another embodiment, the self-emulsifying lipid composition is in the form of a sprayable composition, also referred to as a “spray”. Simply spraying the composition in a hydrophilic phase enables said composition to be dispersed so as to obtain an emulsion, a microemulsion or a nanoemulsion.

According to another embodiment, the dispersion of the self-emulsifying lipid composition in a hydrophilic phase leads to an emulsion, a microemulsion or a nanoemulsion that can be sprayed spontaneously, also called a “spray”.

In another preferred embodiment, the hydrophilic phase according to the invention essentially consists of freshwater or seawater.

The invention relates to a method for preparing the self-emulsifying lipid composition. This method includes the following steps:

an active substance is provided;

an oily phase is provided;

a first surfactant is provided;

a polymer soluble in the composition and/or an ionic polymer insoluble in the composition is provided;

a second surfactant is provided; then

the active substance, the oily phase, the first and the second surfactant, and a soluble polymer and/or an insoluble ionic polymer are mixed under agitation, with or without heating.

The method for preparing the self-emulsifying lipid composition according to the invention advantageously includes the following steps:

a ternary phase diagram is drawn, including the compounds of the self-emulsifying lipid composition and the hydrophilic phase; then

on the basis of said ternary diagram, the proportions of the various compounds of the self-emulsifying lipid composition are selected; then

the self-emulsifying lipid composition is prepared by mixing, with or without heating, under agitation, the various compounds of the self-emulsifying lipid composition.

In a preferred embodiment of the method for preparing said composition, thermal heating may be necessary. In this case, the thermal heating applied is between 30° C. and 50° C., advantageously between 35° C. and 45° C. If it is used, the thermal heating advantageously makes it possible to rapidly solubilize the active substance, and more specifically to fluidize the surfactants of the composition according to the invention.

The ternary phase diagram is established according to techniques well known to a person skilled in the art.

Advantageously, this preparation method is applied to the preparation of a microemulsion or a nanoemulsion.

The invention also relates to a method for preparing an emulsion including the following steps:

a self-emulsifying lipid composition as defined above is provided;

a hydrophilic phase is provided; and

said self-emulsifying lipid composition is added to said hydrophilic phase.

The self-emulsifying lipid composition according to the invention forms electrically charged particles after dispersion in the hydrophilic phase, and is used for the pharmaceutical and/or dermatological treatment of animals. Said composition may also be used for the treatment of plants.

In a preferred embodiment, the self-emulsifying lipid composition according to the invention forms, after dispersion in a hydrophilic phase, positively charged particles for topical application on the skin, coat or appendages of animals. For the administration of a treatment topically or by contact, an insoluble polymer is advantageously used to increase adhesion and prolong the activity. It is preferably chosen from the cationic polymers as described above, so as not to act as a screen between the surface to be treated and the charged particles.

In another embodiment, the self-emulsifying lipid composition according to the invention forms, after dispersion in a hydrophilic phase, negatively charged particles for oral administration of a pharmaceutical and/or nutraceutical treatment in animals. For the oral administration of a treatment, an insoluble polymer is advantageously used and preferably chosen from the anionic polymers as described above, so as to increase the screen between the surface not to be polluted and the charged particles.

The electrically charged particles obtained by dispersion of the self-emulsifying lipid compositions in a hydrophilic phase provide numerous advantages depending on the intended use thereof.

For example, by using drinking water as a vehicle for an oral pharmaceutical or nutraceutical treatment, said negatively charged particles are distributed homogeneously and are stable over time. In addition, these electrically charged particles lessen the harmful effects associated with environmental pollution seen in the prior art. Due to their stability, these negatively charged particles also allow for improved administration of the pharmaceutical or nutraceutical treatment, since there is little to no loss, in the distribution circuits, of the active substance needed for the treatment.

For administration of a treatment by the topical route or by contact, the same observation is made. The positively charged particles are also distributed homogeneously. They are also stable over time, and the harmful effects associated with environmental pollution are limited. The topical administration of the treatment is, again, improved. The positively charged particles, deposited directly onto the surface to be treated or dispersed in an environment where the surfaces to be treated are located, have better adhesion properties. In other words, these particles bind in a particularly stable manner to the surfaces to be treated, leading to an improved distribution of the active substance over time.

Another benefit of the invention is that, regardless of the volume of the hydrophilic phase used, the dispersion of the composition will form an emulsion, a microemulsion, or a nanoemulsion of electrically charged particles.

The self-emulsifying lipid composition according to the invention has the advantage of making it possible to use active substances in solution and in a high content, said active substances generally being known to be water-insoluble. The phrase “high content in solution” means a content greater than the solubility limit of the active substance in the hydrophilic phase. The specific formulation of the self-emulsifying lipid composition according to the invention also makes it possible to obtain a stable solution. This solution is found to be stable for the entire period of preservation of the composition. The self-emulsifying lipid composition according to the invention, forms, after dispersion in the hydrophilic phase, an emulsion, a microemulsion or a nanoemulsion for its entire shelf life. These emulsions, microemulsions or nanoemulsions, due to their preparation at the time of treatment, need to be stable only for the time necessary for application of said treatment, which is a maximum of 24 hours.

### EXAMPLES

Different compositions were prepared and evaluated in applications for therapeutic use in particular for oral or topical administration or by dipping. The amounts described in each of the following examples are indicated as weights with respect to the total weight of the composition. The electric charge of the particles is evaluated by laser doppler microelectrophoresis measuring the zeta potential. An electric field is applied to a dispersion of particles that move at a speed in relation to their zeta potential. This speed can be measured using a laser interferometry technique enabling the electrophoretic mobility to be calculated, then the zeta potential, i.e. the particle charge, to be deduced. The particle size distribution is determined by photon correlation spectroscopy using a dynamic dispersion laser (DLS). The DLS laser analyzes the speed distribution of the particle movement by measuring dynamic variations in the light diffusion intensity caused by the Brownian motion of the particles. This technique gives the diameter of the particles, calculated by the Stokes-Einstein equation on the basis of the aforementioned measurements. The equipment used is a Zetasizer Nano ZS (Malvern Inc.™, Malvern, United Kingdom).

### Example 1

### Composition and Preparation of a Composition Based on Florfenicol Capable of Forming an Emulsion in Water, Said Emulsion Being Intended to be Incorporated in Livestock Drinking Water

TABLE 1
Formulation containing 10 g of florfenicol
per 100 g of composition

| Compounds | % |
|---|---|
| Florfenicol | 10.0 |
| Sodium dioctyl sulfosuccinate in 70% solution | 36.4 |
| in propylene glycol | |
| Polyoxyethylene (20) sorbitan monolaurate | 31.1 |
| Propylene glycol monocaprylate | 12.0 |
| Sodium carboxymethylcellulose | 2.5 |
| N-methyl pyrrolidone | 8.4 |

Florfenicol is introduced and dissolved in the self-emulsifying solution under agitation with heating between 38° C. and 40° C.

The dispersion of the florfenicol composition at 10% by weight was tested at different concentrations covering the mode of use with the four types of dosing pump equipment, namely 1%, 2%, 5% and 10% dosing pumps. These dosing pumps have a small reservoir into which the composition previously emulsified in water is introduced.

To treat pigs from 8 to 200 kg, the florfenicol concentrations must be:

for a 1% dosing pump, between 10 and 16.7 g/l of water, which corresponds to dispersing 100 g to 167 g of the composition according to the invention,

for a 2% dosing pump, between 5 and 8.3 g/l of water, which corresponds to dispersing 50 g to 3 g of the composition according to the invention,

for a 5% dosing pump, between 2 and 3.3 g/l of water, which corresponds to dispersing 20 g to 33 g of the composition according to the invention, and

for a 10% dosing pump, between 1 and 1.65 g/l of water, which corresponds to dispersing 10 g to 16.5 g of the composition according to the invention.

All of the emulsions were obtained by pouring the necessary amount of composition in order to obtain the targeted concentration, under agitation in the volume of water required.

The formulation appears to be dispersible at all of the concentrations, giving homogeneous and more or less turbid dispersions according to the targeted concentration.

All of the dispersions showed perfect stability over more than 24 hours.

The electric charge of the particles as well as their average size were measured with a Zetasizer Nano ZS (Malvern™) for 1 hour.

Procedure followed to obtain the emulsion: in 20 ml of water previously filtered on a 0.22 μm nylon filter, dispersing 1 g of the formulation under agitation at 1000 rpm for 2 minutes.

The measurement of the electric charge is: −70.2±3.4 mV. The average size of the particles is between 350 nm and 750 nm.

The stability, and therefore the homogeneity, of the dispersions shows the repellent effect of the negative charges carried by the particles among one another and their environment.

The active substance is carried in drinking water, and no loss of the active substance in the distribution is observed because no pollution has been detected in the water distribution circuit.

### Example 2

### Composition and Preparation of a Composition Based on Ivermectin Capable of Forming a Microemulsion in Water Intended to be Poured Onto the Backs of Cattle

TABLE 2
Composition containing 5 g of ivermectin per 100 g of formulation

| Compounds | % |
|---|---|
| Ivermectin | 5.0 |
| Caprylocaproyl macrogol-8 glycerides | 38.2 |
| Macrogol-6 oleoyl glycerides | 34.5 |
| Polyglyceryl-3 dioleate | 13.0 |
| Hydroxypropyl guar hydroxypropyltrimonium | 9.3 |
| chloride | |

Ivermectin is introduced and dissolved in the self-emulsifying solution under agitation with heating between 38° C. and 40° C.

The polymer insoluble in the self-emulsifying lipid composition, hydroxypropyl guar hydroxypropyltrimonium chloride, is then progressively dispersed under agitation

The electric charge of the particles as well as their average size were measured with a Zetasizer Nano ZS for 1 hour.

Procedure for obtaining the emulsion: in 20 mm of water previously filtered on a 0.22 μm nylon filter, dispersing 1 g of the formulation under agitation at 1100 rpm for 2 minutes.

The measurement of the electric charge is: +19.2±0.7 mV. The average size of the particles is 15 nm.

The commercial products contain 5 mg of ivermectin per ml. It is simply necessary to disperse, under agitation, 1 g of the composition according to the invention in 10 ml of water in order to obtain the same ivermectin concentration, that is, 1 ml of the microemulsion obtained is equivalent to the 1 ml of the commercial products.

The product is then applied to the dorsolumbar line, from the withers to the base of the tail of cattle, either directly or by using a dosing gun.

It is observed that, once the microemulsion is applied and comes into contact with the skin, the particles literally bind to the latter.

If the description is performed in rain or water is poured after the deposition, the particles are not removed, and the phenomenon of washing away is avoided, thereby providing an optimal treatment for cattle, and no loss of ivermectin to the environment is observed.

### Example 3

### Composition and Preparation of a Composition Based on Deltamethrin, Capable of Forming an Emulsion in WaterIntended to be Vaporized on Fish Farms or Used by Dipping

TABLE 4
Composition B containing 4.7 g of deltamethrin
per 100 g of composition

| Compounds | % |
|---|---|
| Deltamethrin | 4.7 |
| Polyoxyethylene (20) sorbitan monolaurate | 33.3 |
| Lauryl methyl gluceth-10 hydroxypropyldimonium | 30.5 |
| chloride | |
| Propylene glycol monocaprylate | 16.6 |
| Diethylene glycol monoethyl ether | 8.5 |
| Macrogolglycerol ricinoleate | 1.3 |
| Hydroxypropyl guar hydroxypropyltrimonium | 4.9 |
| chloride | |

Deltamethrin is introduced and dissolved in the self-emulsifying solution, previously acidified, under agitation with heating between 38° C. and 40° C.

The polymer insoluable in the self-emulsifying lipid composition, hydroxypropyl guar hydroxypropyltrimonium chloride, is then dispersed in the self-emulsifying solution under agitation with heating between 38° C. and 40° C.

The electric charge of the particles and their average size were measured with a Zetasizer Nano ZS for 1 hour.

Procedure followed to obtain the emulsion: in 50 ml of water previously filtered on a 0.22 μm nylon filter, dispersing 1 g of the composition under agitation at 1000 rpm for 2 minutes.

The measurement of the electric charge is: +23.1±0.9 mV. The average size of the particles is between 200 nm and 250 nm.

The commercical products are concentrated solutions that contain 10 mg of deltamethrin per ml. The concentrated solution is first diluted in an amount of 1 liter in 10 liters of water before being dispersed at the surface of the submerged cages containing the fish. It is simply necessary to disperse, under agitation, 200 g of the composition according to the invention in 10 liters of water to obtain an emulsion having the same deltamethrin concentration as the diluted solution of the commercial products.

One gram of the composition dispersed in 50 ml of water in order to obtain a microemulsion.

Two ml of this microemulsion were poured into 1 m³ tanks containing 50 fish, each around 500 g. Thirty minutes after having poured the emulsions, the water of the tanks was analyzed for deltamethrin, and not trace of the antiparasitic agent was found in each of the tanks.

This demonstrates, owing to the positive changes carried by the particles, that there is no loss of the active molecule to the environment. This observation is extremely important for the treatment of animals as well as for the environment. It is even more significant when the toxicity of deltamethrin for aquatic species and species living in sediment is known. It may be the cause of undesirable effects in the vicinity of the cages, primarily in the sea, containing the aquatic species after the treatment. Deltamethrin is very stable and slowly degradable when it is associated with sediment, under both aerobic and anaerobic conditions. The formulations of the invention minimize or even eliminate the effects of the active molecules on the environment, all while making the entire active molecule available for the treatment.

### Example 4

### Formulations and Preparation of a Colored Formulation Capable of Forming an Emulsion in Water Intended to be Vaporized on Fish

| | In |
|---|---|
| Formulation A | w/w % |
| 1,4-bis(ethylamino)-9,10-anthraquinone* | 50 mg |
| Polyoxyethylene (20) sorbitan monolaurate | 37.5 |
| Lauryl methyl gluceth-10 hydroxypropyldimonium | 35.5 |
| chloride | |
| Propylene glycol monocaprylate | 20.0 |
| Polyoxyl 35 castor oil | 2.0 |
| Dimethylaminoethyl methacrylate, butyl | 5.0 |
| methacrylate, and methyl methacrylate copolymer | |

*oil-soluble, water-insoluble blue dye

The dye is introduced and dissolved in the self-emulsifying solution under agitation with light heating.

The “soluble” copolymer, dimethylaminoethyl methacrylate, butyl methacrylate, and methyl methacrylate, is then introduced and dissolved in the self-emulsifying solution under agitation with light heating.

The electric charge of the particles as well as their average size were measured with a Zetasizer Nano ZS™.

Procedure followed to obtain the emulsion: in 50 ml of water previously filtered on a 0.22 μm nylon filter, dispersing 1 g of the formulation under agitation at 1000 rpm for 2 minutes.

The measurement of the electric charge is: +43.7±0.8 mV. The average size of the particles is between 200 nm and 700 nm.

Two grams of the composition are dispersed in 50 ml of water in order to obtain a microemulsion.

The water circulation system in the aquarium is stopped, the microemulsion is poured in the center of a 250-1 aquarium containing 30 rainbow trout, each around 50 g. Thirty minutes after having poured the emulsion, 10 fish are anesthetized, and an examination of the surface of the body of said fish is performed under microscope.

In parallel, a plastic sheet is placed on the floor aquarium floor, 1 hour after having poured the emulsion, the plastic sheet is removed from the aquarium, and an examination of the surface of the sheet is performed under microscope.

While microparticles of the emulsion are observed at the surface of the skin of the fish, no particle is visible on the surface of the sheet.

This demonstrates, owing to the positive charges carried by the particles, that there is no loss of microparticles to the environment, and that, in particular, they all adhered to the skin of the fish.

1. A self-emulsifying lipid composition capable of self-emulsifying in the presence of a hydrophilic phase, including:
from 0.001% to 20% by weight, relative to the total weight of the composition, of at least one active substance;
from 5% to 60% by weight, relative to the total weight of the composition, of an oily phase;
a first surfactant;
a second surfactant;
from 0.5% to 20% by weight with respect to the total weight of the composition of at least one polymer soluble in the composition and/or an ionic polymer insoluble in the composition;
wherein a ratio between the first surfactant and the second surfactant is between 0.5 and 6; and
wherein the lipid composition is so that, after dispersion in a hydrophilic phase, it forms particles having a charge having an absolute value in a range of from 10 mV to 100 mV.

2. The self-emulsifying lipid composition according to claim 1, wherein the active substance is chosen from the group consisting of anti-infectious agents; cardiotonics, antiparasitic agents, insecticides, insect growth inhibitors; anti-arthritic agents; steroidal and nonsteroidal anti-inflammatories; antihistamines; hormones; digestive therapy substances; respiratory therapy substances; substances acting on the nervous system macroelements, microelements and trace elements; vitamins; plant and animal organ extracts, and mixtures thereof.

3. The self-emulsifying lipid composition according to claim 1, wherein each of the first surfactant and the second surfactant is chosen from the group consisting of:
anionic surfactants;
cationic surfactants; and
non-ionic surfactants.

4. The self-emulsifying lipid composition according to claim 1, wherein the oily phase is chosen from the group consisting of plant-based oils saturated or unsaturated fatly acid esters; fatty alcohol ethers; mixtures of mono-, di-, and triglycerides including a fatty acid with 8 to 18 carbon atoms and polyethylene glycol diesters; saturated and unsaturated fatty acid esters, and propylene glycol.

5. The self-emulsifying lipid composition according to claim 1, wherein the polymer is chosen from the group consisting of:
anionic polymers;
cationic polymers; and
non-ionic polymers.

6. The self-emulsifying lipid composition according to claim 1, wherein the polymer soluble in the composition and/or the ionic polymer insoluble in the composition is/are present in a proportion in a range of from 1% to 15% by weight of the total weight of the composition.

7. The self-emulsifying lipid composition according to claim 1, wherein the composition also includes at least one additive selected from the group consisting of hydrophilic solvents, preservatives, sweetening agents and appetizing agents.

8. The self-emulsifying lipid composition according to claim 7, wherein the additives are present in a proportion in a range of from 0.01% to 30% by weight of the total weight of the composition.

9. A method for preparing a self-emulsifying lipid composition, including:
providing an active substance;
providing an oily phase;
providing a first surfactant;
providing a polymer soluble in the composition and/or an ionic polymer insoluble in the composition;
providing a second surfactant; then
mixing the active substance, the oily phase, the first surfactant, the second surfactant, and the soluble polymer and/or the insoluble ionic polymer under agitation, with or without heating,
so as to obtain the self-emulsifying lipid composition according to claim 1.

10. The method according to claim 9, including, prior to mixing:
drawing a ternary phase diagram including the compounds of the self-emulsifying lipid composition and the hydrophilic phase; then
selecting, on the basis of the ternary diagram, the proportions of the compounds of the self-emulsifying lipid composition; then
preparing the self-emulsifying lipid composition by mixing, with or without healing, under agitation, the compounds of the self-emulsifying lipid composition.

11. An emulsion for the pharmaceutical and/or dermatological treatment of animals, comprising the sell-emulsifying lipid composition according to claim 1 and a hydrophilic phase, wherein the self-emulsifying lipid composition forms electrically charged particles dispersed in the hydrophilic phase.

12. A method of providing a nutraceutical treatment lo an animal, comprising administering to the animal an emulsion comprising the self-emulsifying lipid composition according to claim 1 and a hydrophilic phase, wherein the self-emulsifying lipid composition forms electrically charged particles dispersed in the hydrophilic phase.

13. A method of treating plants, comprising administering to the plant an emulsion comprising the self-emulsifying lipid composition according to claim 1, wherein the self-emulsifying lipid composition forms electrically charged panicles dispersed in the hydrophilic phase.

14. An emulsion for topical application on the skin, coat or appendages of animals in the treatment of a disease, comprising the self-emulsifying lipid composition according to claim 1 and a hydrophilic phase, wherein the self-emulsifying lipid composition forms positively charged particles dispersed in the hydrophilic phase.

15. An emulsion for oral administration of a pharmaceutical treatment in animals, comprising the lipid composition according to claim 1 and a hydrophilic phase, wherein the self-emulsifying lipid composition forms negatively charged particles dispersed in the hydrophilic phase.

16. An emulsion for oral administration of a nutraceutical treatment of animsl, comprising, the lipid composition according to claim 1 and a hydrophilic phase, wherein the self-emulsifying lipid composition forms negatively charged particles dispersed in the hydrophilic phase.

17. The self-emulsifying lipid composition according to claim 2, wherein the active substance is chosen from the group consisting of antibiotics and sulfamides as anti-infectious agents; cardiotonics, antiparasitic agents; insecticides, insect growth inhibitors; anti-arthritic agents, steroidal and nonsteroidal anti-inflammatories; antihistamines, prostaglandins as hormones; gastrointestinal dressings and sedatives, antiulcer agents and replacement flora, anti-diarrheal agents, hepatoprotective agents, antispasmodic agents, laxatives, intestinal antiseptics as digestive therapy substances; respiratory analeptics, cough suppressants, bronchodilators, mucolytics, and respiratory antiseptics as respiratory therapy substances; analgesics, sedatives and tranquilizers as substances acting on the nervous system, macroelements, microelements and trace elements, vitamins; plant and animal organ extracts; and mixtures thereof.

18. The self-emulsifying lipid composition according to claim 3, wherein each of the first surfactant and the second surfactant is chosen from the group consisting of:
as anionic surfactants, alkyl sulfates, alkyl ether sulfates, amidoalkyl ether sulfates, aryl alkyl polyether sulfates, monoglyceride sulfates, alkyl sulfonates, amidoalkyl sulfonates, aryl alkyl sulfonates, alpha olefin sulfonates, paraffin sulfonates, alkyl sulfosuccinates, ether alkyl sulfosuccinates. amidoalkyl sulfosuccinates, alkyl sulfoacetates, acyl sarcosinates, acyl glutamates, alkyl sulfosuccinamates, acyl thionates, N-acyl laurate, alkyl monoester salts and polycarboxylic polyglycoside acids, acyl lactylates, D-galactoside uronic acid salts, carboxylic alkyl ether acid salts, carboxylic alkyl aryl ether acid salts, carboxylic alkyl ether amido acid salts;
as cationic surfactants, amine ethoxylates; amine salts, protein derivatives, quaternary ammonium salts, and
as non-ionic surfactants, hydrogenated castor oil poly ethers and ethylene oxide; fatty acid and sorbitan esters: polyoxyethylated fatty acid and sorbitan esters; fatty acid and polyethylene glycol esters; fatty acid and polyoxylglyceride esters, polyglycerol fatty acid esters.

19. The self-emulsifying lipid composition according to claim 4, wherein the oily phase is chosen from the group consisting of:
as plant-based oils, palm oil, sunflower oil, rapeseed oil, castor oil, peanut oil, and corn oil;
as saturated or unsaturated fatty acid esters, fatty acids with 8 to 18 carbon atoms and including hydrocarbon chains with 1 to 12 carbon atoms,
as fatty alcohol ethers, distearyl ether, triglycerides, diglycerides, monoglycerides and mixtures thereof;
as mixtures of mono-, di-, and triglycerides, fatty acids with 8 to 18 carbon atoms and polyethylene glycol diesters;
as saturated or unsaturated fatty acid esters, fatty acids with 8 to 18 carbon atoms and propylene glycol.

20. The self-emulsifying lipid composition according to claim 5, wherein the polymer is chosen from the group consisting of:
as anionic polymers, anionic derivatives of starch, anionic derivatives of cellulose;
as cationic polymers, copolymers of dimethylaminoethyl methacrylate, butyl methacrylate, and methyl methacrylate, copolymers of vinylpyrrolidone and substituted quaternized vinylpyrrolidone, butyl acrylate chloride/ethyltrimonium/styrene, poly(diallyldimethylammonium) chloride, acrylamide and diallyldimethylammonium chloride copolymers, hydroxy propyl guar hydroxypropyltrimonium chloride, guar hydroxypropyltrimonium chloride, modified xanthan gums, chitosan and derivatives thereof; and
as non-ionic polymers, cellulose ethers.
